(19) United States
(12) Patent Application Publication
(10) Pub. No.: US 2025/0380975 A1
(43) Pub. Date: Dec. 18, 2025

(54) SCREW ELEMENT FOR USE IN SPINAL, ORTHOPAEDIC, OR TRAUMA SURGERY

(71) Applicants: Lutz BIEDERMANN, VS-Villingen (DE); Timo BIEDERMANN, Trossingen (DE); Bernd FISCHER, Friedenweiler (DE)

(72) Inventors: Lutz BIEDERMANN, VS-Villingen (DE); Timo BIEDERMANN, Trossingen (DE); Bernd FISCHER, Friedenweiler (DE)

(21) Appl. No.: 19/222,012

(22) Filed: May 29, 2025

Related U.S. Application Data
(60) Provisional application No. 63/659,525, filed on Jun. 13, 2024.

(30) Foreign Application Priority Data
Jun. 13, 2024 (EP) 24 182 099.2

(51) Int. Cl. A61B 17/86; A61B 17/70; A61B 17/80; B22F 10/28; B33Y 10/00; B33Y 80/00

(52) U.S. Cl. CPC A61B 17/8615; A61B 17/866; B22F 10/28; B33Y 10/00; B33Y 80/00; A61B 17/7034; A61B 17/8052; B22F 2998/10

(57) ABSTRACT

A screw element for use in spinal, orthopaedic, or trauma surgery, has a screw axis, a screw thread, and a drive portion including a wall that defines an axially extending recess for engaging a tool. The recess has a cross-sectional profile that extends from a first end to a second end in a direction of the screw axis. The cross-sectional profile has a first portion positioned closest radially to the screw axis and a plurality of drive grooves that extend radially away from the first portion and that are spaced apart from each other in a circumferential direction around the screw axis. A portion of the wall includes a protrusion that protrudes laterally away from the wall into the recess. The protrusion is spaced apart axially from the first and second ends of the recess.

### CROSS-REFERENCE TO RELATED APPLICATION(S)

The present application claims priority to and the benefit of U.S. Provisional Patent Application No. 63/659,525, filed Jun. 13, 2024, the contents of which are hereby incorporated by reference in their entirety, and claims priority from European Patent Application EP 24 182 099.2, filed Jun. 13, 2024, the contents of which are hereby incorporated by reference in their entirety.

### BACKGROUND

### Field

The present application relates to a screw element for use in spinal, orthopaedic, or trauma surgery. In particular, the screw element can be a screw element for a bone anchoring device configured to be connected to a rod, a screw element for a bone plate, or a set screw configured to be used with such devices.

In spinal, orthopaedic, or trauma surgery, the insertion of screw elements into bone or the use of screw elements such as a set screw requires precision. Often, either the screw element is a relatively tiny element, or the available space for handling the screw element is reduced. Therefore, it is important to avoid loss of the screw element in a patient's body and/or to provide an effective way to safely tighten the screw element.

### Description of Related Art

U.S. Pat. No. 9,867,639 B2 describes a screw element for use in spinal, orthopaedic, or trauma surgery, the screw element including a screw axis and a screw thread, and a drive portion for engagement with a screwdriver, wherein the drive portion has a first recess with a wall and substantially longitudinally extending drive grooves formed in the wall, a second recess between a free end of the screw element and the first recess, wherein the second recess has a wall and a gradually increasing inner diameter towards the free end, and wherein in the wall of the second recess guide grooves are formed at positions corresponding to the position of the drive grooves that allow an engagement portion of a screwdriver to be guided to engage the drive grooves. This screw element is, for example, useful in minimally invasive surgery as it facilitates the insertion of the screwdriver in the drive portion.

### SUMMARY

There is still a need for further improving the handling of a screw element with a screwdriver, in particular in view of the above-mentioned clinical applications.

It is therefore an object of the invention to provide a screw element, and a system including a screw element and a screwdriver, that facilitates easier handling. It is also an object to provide a method for manufacturing such a screw element.

According to an aspect of the invention, a screw element for use in spinal, orthopaedic, or trauma surgery is provided, the screw element including a screw axis, a screw thread, and a recessed drive portion with a wall that defines a main contour configured to engage with a screwdriver, the wall further defining a plurality of drive grooves, wherein the drive grooves are spaced apart from each other in a circumferential direction around the screw axis, and wherein a portion of the wall has a protrusion that protrudes from the main contour into the recess.

The main contour may be considered, for example, the wall portions which correspond to the shape of the engagement portion of a screwdriver so that a form-fit connection can be established.

The screw element provides for an enhanced clamping of the engagement portion of a screwdriver. This allows a safe and more secure holding of the screw element when the screw element is screwed into bone or when the screw element is used as a set screw.

Moreover, the screw element allows for application of force more efficiently to screw in the screw element, since radial play between the engagement portion of the screwdriver and the drive portion of the screw element may be reduced.

The drive portion can have various shapes, such as a polygonal shape or a star-shape (e.g., a torx-shape), or any other shape suitable for providing a form fit engagement with an engagement portion of a screwdriver.

In another aspect of the invention, the screw element may be used as part of a polyaxial bone anchoring device. The polyaxial bone anchoring device may include the screw element and a receiving part in which a head of the screw element is pivotably held, and where the screw element can be locked with the screw axis forming an angle relative to a central axis of the receiving part. On the head of the screw element, the drive portion with the protrusion formed on a wall portion of the drive portion may be provided. The receiving part further has a recess for receiving a rod. The rod can be fixed relative to the receiving part by means of a screw element in the form of a set screw, which may include a drive portion with a protrusion on its wall portion.

In another aspect of the invention, the screw element may be used together with a bone plate, where the screw element is configured to fix the bone plate to bone. On the head of the screw element, a drive portion with a protrusion on a wall portion may be provided. The head of the screw element may be held in a seat provided around a through-hole in the bone plate, through which the shank of the screw element extends. The head may be secured against being pulled out of the seat by a screw element in the form of a set screw which may include a drive portion with a protrusion on its wall.

According to a further aspect of the invention, the screw element is manufactured with an additive manufacturing technique, such as laser sintering, laser melting, electron beam melting, fused deposition molding (FDM), or others. With such a technique, any three-dimensional shape can be manufactured based on CAD-data of the screw element, especially any complicated shapes that cannot be manufactured with subtractive methods or that would be difficult to manufacture with subtractive methods. The manufacturing can be carried out on demand for a specific patient's application.

At least a portion of the screw element that includes the drive portion with the protrusion on a wall portion may have a size and a shape that can most efficiently only be manufactured by an additive manufacturing method.

The use of an additive manufacturing method allows to more easily design various screw elements with different protrusion shapes and/or arrangements. By means of this, various holding forces between the engagement portion of the screwdriver and the drive portion of the screw element can be achieved, for example.

### BRIEF DESCRIPTION OF THE DRAWINGS

Further features and advantages will become apparent from the description of embodiments by means of the accompanying drawings. In the drawings:

FIG. 1 shows a perspective view of a screw element according to a first embodiment, together with a front portion of a screwdriver, prior to attaching the screwdriver to the screw element.

FIG. 2 shows an enlarged view of a portion of FIG. 1.

FIG. 3 shows a cross-sectional view of a portion of the screw element of FIGS. 1 and 2, including a head of the screw element, and an engagement portion of the screwdriver of FIGS. 1 and 2, the cross-section taken in a plane including a screw axis of the screw element and an axis of rotation of the screwdriver.

FIG. 4 shows a cross-sectional view of the screw element and the engagement portion of FIGS. 1 to 3 with the engagement portion of the screwdriver engaging the screw element, the cross-section taken in the same plane as seen in FIG. 3.

FIG. 5 shows an enlarged portion of FIG. 4.

FIG. 6 shows another perspective view of a portion of the screw element of FIGS. 1 to 5, the view including the head of the screw element.

FIG. 7 shows a perspective view of a modified first embodiment of the screw element, the view including the head of the screw element.

FIG. 8 shows a perspective view of another modified first embodiment of the screw element, the view including the head of the screw element.

FIG. 9 shows a perspective view of a further modified first embodiment of the screw element, the view including the head of the screw element.

FIG. 10 shows a perspective view of a polyaxial bone anchoring device utilizing a second embodiment of the screw element in the form of a set screw.

FIG. 11 shows an enlarged perspective view of the second embodiment of the screw element from FIG. 10.

FIG. 12 shows a perspective view of a modified second embodiment of the screw element.

FIG. 13 shows a perspective view of a further modified second embodiment of the screw element.

FIG. 14 shows a perspective view of an application of the screw elements according to the first embodiment and the modified first embodiments as shown in FIGS. 1 to 9.

FIG. 15 shows a perspective view of another application of the screw elements according to the first embodiment and the modified first embodiments shown in FIGS. 1 to 9 and an application of the screw elements according to the second embodiment and the modified second embodiments shown in FIGS. 10 to 13.

FIG. 16 shows a still further application of the screw elements according to the first embodiment and the modified first embodiments shown in FIGS. 1 to 9.

### DETAILED DESCRIPTION

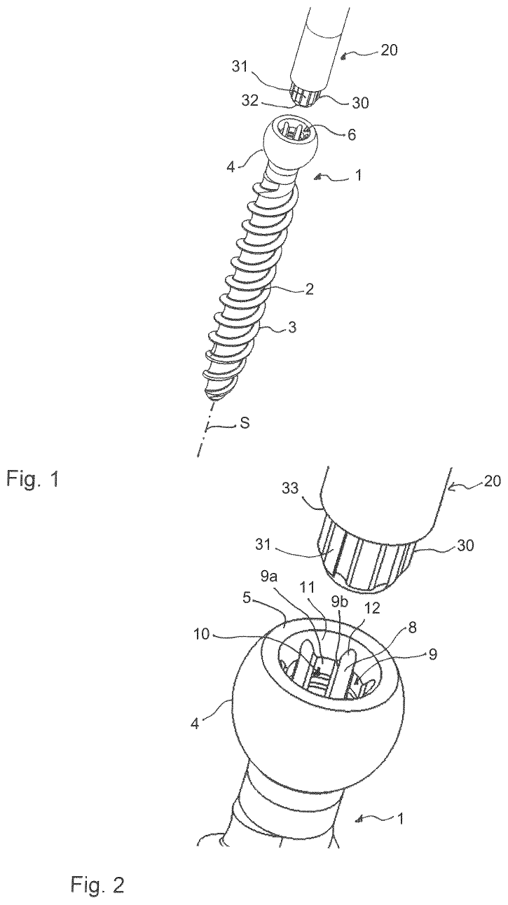

Referring to FIGS. 1 to 6, a screw element 1 according to a first embodiment includes a shank 2, with a bone thread (or more generally a screw thread) 3 on at least a portion of the shank 2, and a head 4. The shank 2 is configured to be inserted into a bone, for example, into a pedicle of a vertebra. A screw axis S is defined by the axis of the bone thread 3. The head 4 has a spherical segment shape and a free end 5 on a side opposite the shank 2. A drive portion 6 that is configured to engage with an engagement portion 30 of a screwdriver 20 is provided at the free end 5.

The drive portion 6 of the screw element 1 has a substantially cylindrical first recess 7 that is located at a distance from the free end 5 and that is surrounded circumferentially by an inner wall 9 defining a main contour suitable for engagement with the screwdriver. The wall is also substantially cylindrical with a main inner diameter and with a cylinder axis that is coaxial with the screw axis S. A plurality of longitudinal drive grooves 8 are defined by the inner wall. A cross-section of each of the drive grooves 8, taken along a plane perpendicular to the screw axis S, may be substantially circular segment-shaped or otherwise rounded. From a top view, the drive grooves 8 are arranged circumferentially around the first recess 7 in a star-like manner. For example, an even number, such as six, drive grooves 8 may be provided. In addition, the first recess 7 has a substantially flat bottom 7a. An axial depth of the first recess 7 is such that the engagement portion 30 of the screwdriver can be immersed or inserted therein. In particular, the depth of the first recess may substantially correspond to an axial length of the engagement portion 30 of the screwdriver 20. Thus, the size of the first recess 7 with the drive grooves 8 is sufficient for applying a necessary torque for inserting or advancing the screw element 1.

Between consecutive drive grooves 8 in a circumferential direction, there are wall portions 9a. In the embodiment shown, the wall portions 9a have a cylindrical shape due to the cylindrical main contour of the first recess 7. The drive grooves 8 may merge into the cylindrical wall portions 9a via slanted portions 9b.

On each wall portion 9a, a protrusion 10 is formed, that protrudes into the recess 7. In other words, the protrusion 10 protrudes, e.g., radially inwardly, from the main contour defined by the wall. The protrusion 10 in this embodiment includes two axially spaced apart circumferentially extending ribs 10a, 10b that extend entirely across each of the wall portions 9a in the circumferential direction. An axial position of the ribs 10a, 10b may be substantially at or around the center of each of the wall portions 9a. A height of the protrusion 10 above the main contour (i.e., a radial extension of the protrusion 10) is such that the protrusion 10 permits insertion of the engagement portion 30 of the screwdriver 20 but reduces radial play of the engagement portion 30 when the engagement portion is inserted into the first recess 7. In addition, the protrusion 10 has a size and/or shape such that the protrusion can hold the engagement portion 30 in the first recess 7 by friction. In a practical example, the height to which the protrusion protrudes above or inwardly from the main contour of the wall portion 9a may only be a few hundredths of a millimeter. In the embodiment shown, each of the wall portions 9a has a protrusion 10, however, it may be sufficient that at least one of the wall portions 9a, preferably more than one wall portion, and still more preferably wall portions 9a that are arranged opposite to each other, have the protrusion 10. The shape of the ribs 10a, 10b is rounded, in particular, free of any sharp edges, such that the insertion of the engagement portion 30 can be effected smoothly, in particular, without jamming.

Between the first recess 7 and the free end 5, there may be a second recess 11 that tapers, e.g., conically, and narrows from the free end 5 towards the first recess 7. A depth of the second recess 11 in the axial direction may be approximately one fifth to one third of the depth of the first recess 7. The second recess 11 provides an enlarged bevelled surface that facilitates insertion of the engagement portion 30 of the screwdriver 20 into the drive portion 6. A plurality of guide grooves 12 are provided in the wall defining the second recess 11 at positions corresponding to the positions of the drive grooves in the first recess 7. The guide grooves 12 have a size and a shape such that the guide grooves merge into the drive grooves 8. Due to the bevelled surface of the second recess 11, the depth of the guide grooves 12 gradually increases from the free end 5 towards the drive groove 8. This allows for a more precise guiding of the engagement portion 30 of the screwdriver 20 into the first recess 7, while simultaneously facilitating the engagement between the engagement portion 30 and the drive portion 6.

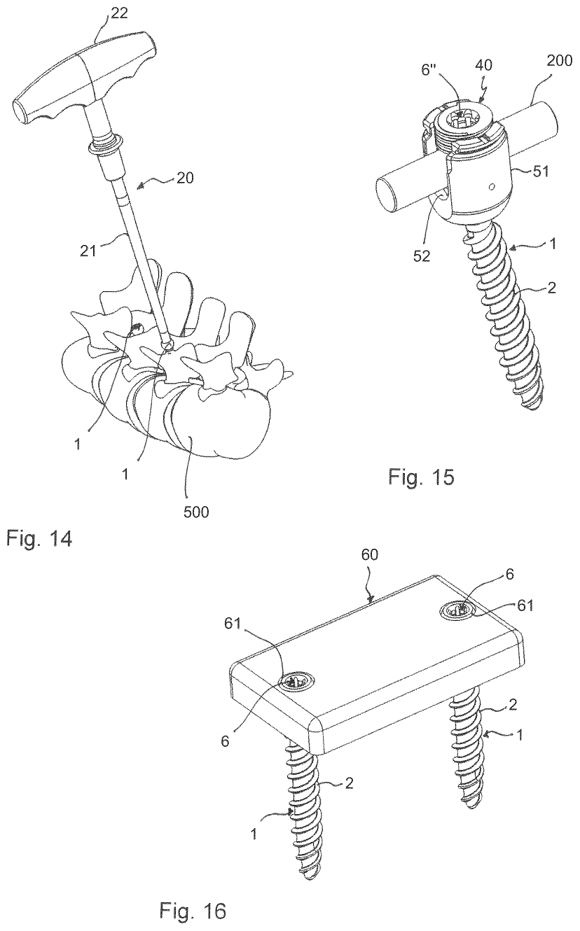

The screwdriver includes a drive shaft 21, a handle 22 (see, e.g., FIG. 14) at one end of the drive shaft 21, and the engagement portion 30 at the opposite end of the drive shaft 21. The engagement portion 30 has a substantially cylindrical main contour that fits into the first recess 7. In addition, the engagement portion has longitudinally extending rib-like engagement protrusions 31 that are sized to engage the drive grooves 8 to apply torque to the screw element 1. The engagement portion may be bevelled towards a free end surface 32 of the engagement portion 30. Moreover, between the engagement portion 30 and the shaft portion, there may be a shoulder 33 for abutting against the free end 5 of the head 4.

The screw element may be made of any bio-compatible material, preferably, however, of titanium or stainless steel, or of any other bio-compatible metal or metal alloy or plastic material. With respect to bio-compatible alloys, a NiTi-alloy, for example Nitinol, may be used. Other materials that can be used can also be magnesium or magnesium alloys. Bio-compatible plastic materials that can be used may be, for example, polyether ether ketone (PEEK) or poly-L-lactide acid (PLLA).

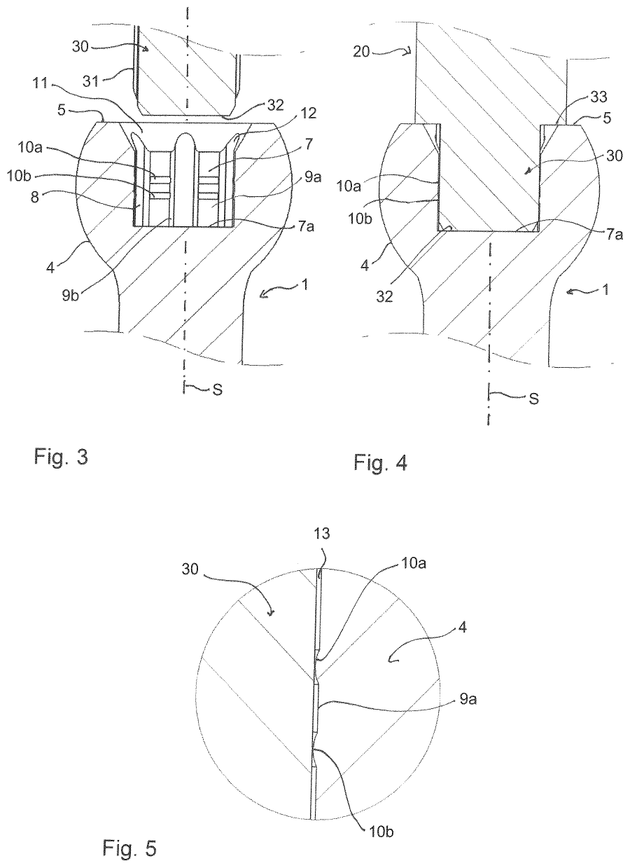

In use, as depicted in FIGS. 3 to 5, when the engagement portion 30 is inserted into the drive portion 6, the engagement portion enters the first recess 7 until the free end surface 32 abuts against the bottom 7a of the first recess 7. It should be noted that in FIG. 3, the engagement portion is oriented with its drive axis coaxial with the screw axis. If the drive axis is not coaxial with the screw axis prior to the engagement portion entering the recess 7, the guide grooves 12 guide the engagement portion 30 so that the engagement portion aligns with the first recess 7 during insertion. As can be seen in FIGS. 4 and 5, there is a radial gap 13 between the outer surface of the engagement portion 30 and the wall portions 9a, except at the positions of the protrusion 10, i.e., at the positions of the ribs 10a, 10b. Hence, the protrusion 10 reduces the radial play and clamps the engagement portion so that the engagement portion is firmly held in the first recess 7 by friction. By means of this, the screw element 1 can be held more safely and securely before the screw element is inserted into bone. During screwing in of the screw element 1, the force applied to the screw element can also be transferred more efficiently.

In particular, the screw element as a whole, or at least a portion including the drive portion 6, is manufactured using an additive manufacturing method, more specifically, an additive layer-manufacturing method. In such a method, the screw element is built up by layer-wise deposition of a building material, and the building material in each layer is melted or otherwise solidified at positions corresponding to the cross-section of the screw element in the respective layer. A suitable method is, for example, selective laser-sintering (SLS) or selective laser-melting (SLM), in which the building material is a powder, such as a metal powder or a plastic powder, and a laser is used to melt the powder. In this method, consecutive layers of the building material are applied to a support surface or a previous layer, and the laser beam is steered to the positions of a layer which correspond to the cross-section of the screw element in each layer. Thus, the screw element is built up in an additive manner. The building material may preferably be a titanium powder or a stainless-steel powder. Alternatively, an electron beam may be used to melt the building material. Also, other known methods of powder-based three-dimensional printing, in which layers of a powder material are deposited and solidified by applying a binder material at positions corresponding to the screw element, may be used. Still further additive manufacturing methods, for example, fused deposition modelling (FDM), may also be used.

With these methods, it is possible to produce the screw element having the protrusions 10 in the drive portion 6. It should be mentioned that the screw element 1 can also be produced as a two-part device, wherein, for example, the shank is produced conventionally on a lathe and using a thread-cutting device, whereas the head with the drive portion 6 is produced via an additive manufacturing method as described above. The two parts can then be assembled, for example, by means of a press-fit connection or a threaded connection, among other assembly methods.

It shall be noted that an additive manufacturing method, in particular an additive layer-manufacturing method, may affect the outer appearance of the screw element. For example, the layers may be visible on the surface of the finished object even if it is post-treated, such as polished, etched, coated, or otherwise treated. It may also be possible to identify the traces of the laser or electron beam when inspecting the fabricated object. Hence, the use of an additive manufacturing method, in particular the additive layer-manufacturing method, can be distinguished based on the finished object, when compared for example to a conventional subtractive manufacturing method.

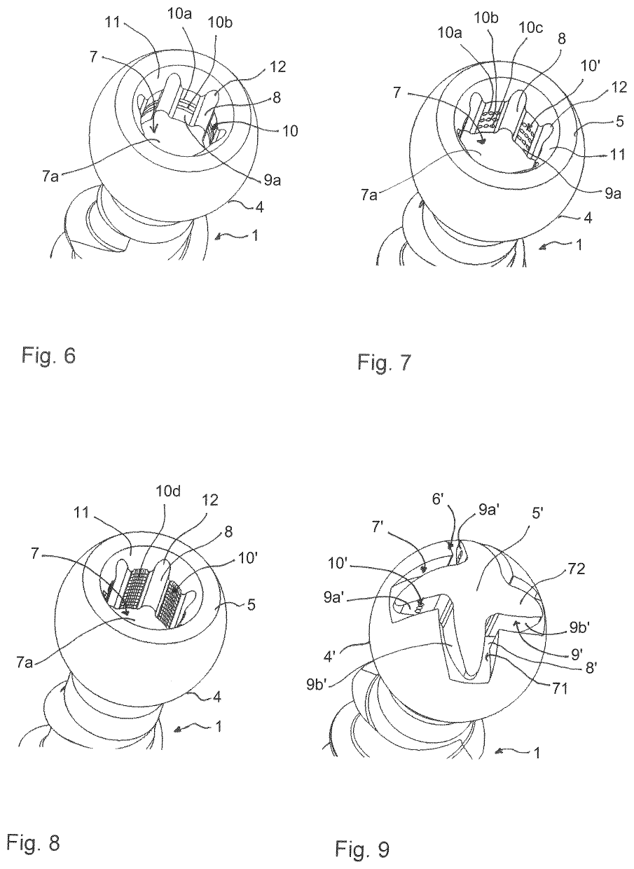

FIG. 6 depicts the screw element according to the first embodiment from another perspective view. In particular, the bottom 7a of the first recess is shown.

Referring further to FIGS. 7 to 9, various modifications of the first embodiment of the screw element are shown. Parts and portions that are identical or similar to the first embodiment of the screw element shown in FIGS. 1 to 6 are marked with the same reference numerals, and the description thereof will not be repeated.

A modification according to FIG. 7 differs in the design of the protrusion. The protrusion 10′ includes at least one, and preferably more than one, circumferentially extending rows of pimples or small bulges. Rows 10a, 10b, 10c of pimples or bulges extend along the full width of a wall portion 9a in the circumferential direction. The number and size of the pimples and the number of rows may be selected according to a desired holding force. It shall be noted that the lowermost row may be located close to the bottom 7a of the first recess 7. The uppermost row may be located at a distance from the upper edge of the wall portion 9 to facilitate insertion of the engagement portion 30.

A further modification is shown in FIG. 8, where the protrusion 10′ includes a structured area of small projections 10d. The projections 10d can be a protruding area of local roughness, or can be a field of pimples or rounded pyramids or other bulges that are arranged close to each other, for example, that may be regularly arranged in an array-like manner.

A further modification of the screw element is shown in FIG. 9. The embodiment shown in FIG. 9 differs from the previous modifications by the design of the drive portion. The drive portion 6′ in FIG. 9 has a spiral shape, similar to a drive known under the name Mortorq®. The screw element 1 according to this modified embodiment includes a spherical head 4′ that also has a spherical outer surface at a free upper end 5′. In the embodiment of FIG. 9, the head is substantially entirely spherical. A recess 7′ for the screwdriver is formed by a groove 71 provided in the spherical surface of the head 4′ in a manner such that an inner wall 9′ formed by the groove 71 gives the upper head portion a shape of arms 72 that are bent in a spiral-like manner around a center of the upper free end 5′. In the embodiment shown, the recess 7′ includes four arms 72. The sections of the groove 71 forming the arms 72 also provide the drive grooves 8′. Each arm 72 includes a first wall portion 9a′ and an opposite second wall portion 9b′, wherein the first wall portion 9a′ may be slightly concavely formed or substantially flat and the second wall portion 9b′ may be convexly formed, thus providing the spiral-like shape of the arm 72. On the first wall portion, the protrusion 10′ is provided, preferably substantially in the center of the wall portion 9a′ in a lateral direction. The protrusion 10′ may include, for example, several pimples or bulges that are arranged in a vertical line in the axial direction of the screw element. However, other arrangements of the protrusion 10′ may also be selected and incorporated.

The corresponding engagement portion (not shown) of the screwdriver has a shape that fits into the groove 71. By means of this shape, an engagement surface between the engagement portion of the screwdriver and the drive portion of the screw element can be enhanced, and thus the engagement may be more robust, when compared to usual polygon engagement shapes. Therefore, the loads that can be transferred onto the head 4′ of the screw element in FIG. 9 may be comparatively higher.

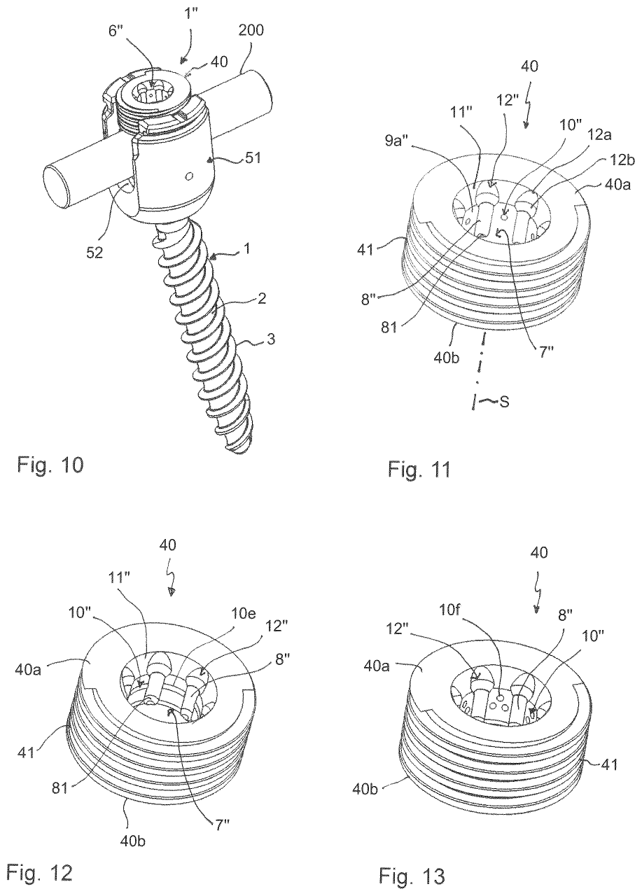

A second embodiment of a screw element is explained with reference to FIGS. 10 and 11. Parts and portions that are the same or substantially the same as in the previous embodiments are marked with the same reference numerals, and the descriptions thereof will not be repeated. In the embodiment of FIGS. 10 and 11, the screw element 1″ is formed as a set screw 40 that is used in a polyaxial bone anchoring device. The polyaxial bone anchoring device is shown only in an exemplary manner, and it should be understood that many different designs of such polyaxial bone anchoring devices may be contemplated without departing from the spirit and scope of the invention.

In greater detail, the polyaxial bone anchoring device includes a bone anchor, which may be in the form of the screw element 1 with the shank 2, a spherical segment-shaped head (not shown), and a drive portion 6, 6′ on the head, e.g., according to the previously described first embodiment and/or its respective modifications. However, a bone anchor with a conventional drive portion such as a torx-shaped drive portion or a polygon-shaped drive portion in the head may also be used. The screw element 1 is pivotably connected to a receiving part 51 that includes a seat to hold the head of the screw element 1, for example, in a ball and socket manner. A pressure element (not shown) may also be provided to exert pressure onto the head. The receiving part 51 also includes a substantially U-shaped recess 52 that is configured to receive a rod 200 therein. The rod 200 may be connected to a plurality of bone anchoring devices. To lock the rod 200 in a receiving part 51 and a pivot position of the head relative to the receiving part 51, the screw element 1″ according to the second embodiment, which is in the form of the set screw 40, can be used.

The set screw 40 has a first end or upper end 40a and a second end or lower end 40b. An external thread 41 of the set screw 40 cooperates with a corresponding internal thread on the receiving part 51. At a distance from the first end 40a, a first recess 7″ is formed. The first recess 7″ forms substantially cylindrical inner wall portions 9a″ that are separated from each other by longitudinal drive grooves 8″, the bottom 81 of which provides an abutment for the drive portion 30 of the screwdriver 20 in the axial direction. The bottom of the recess 7″ in some embodiments may be open towards the lower end 40b. The second recess 11″ may be slanted or tapered like in the first embodiment. At positions corresponding to the positions of the drive grooves 8″, guide grooves 12″ are formed in the slanted second recess 11″. The guide grooves 12″ may have a greater width than the corresponding drive grooves 8″, respectively. In greater detail, the guide grooves 12″ may have a first groove portion 12a adjacent to the upper end 40a that is rounded and that has a gradually increasing width as the guide groove extends in the direction of the first recess 7″. Furthermore, the guide grooves 12″ may include an intermediate section in the form of an inclined shoulder 12b that narrows towards the drive grooves 8″. The intermediate section 12b and the first portion 12a of the guide grooves may form pocket-like recesses that catch and guide the engagement portion 30 of the screwdriver 20 into the guide grooves 8″.

On the wall portions 9a″, a protrusion 10″ is provided. In the embodiment, the protrusion 10″ may include a single bulge or a single pimple provided substantially at the center of each wall portion 9a″ in the circumferential direction. The protrusion 10″ may be closer to the second recess 11″ than to the bottom 81 of the drive grooves 8″ in the axial direction. However, the protrusion 10″ can also be, for example, in the middle of the wall portion 9a″ in an axial direction or at any other axial position.

In use, the screw element 1″ in the form of the set crew 40 is engaged by the engagement portion 30 of the screwdriver 20. By means of the protrusion 10″, the engagement portion is firmly clamped in the first recess 7″. This more easily facilitates the insertion of the screw element 1″ into the U-shaped recess 52 of the receiving part 51 of the polyaxial bone anchoring device, and may further improve the transfer of force to tighten the set screw 40. Once the head of the bone anchor and the rod are locked, further adjustments may become necessary. To make such adjustments, the set screw 40 can be loosened and tightened again after correcting the angular position of the receiving part 51 relative to the head and/or after correcting the position of the rod 200.

FIG. 12 shows a modification of the screw element 40, wherein the protrusion 10″ is formed as a circumferential rib 10e that extends entirely across at least one of the wall portions 9a″ in the circumferential direction.

In a further modification shown in FIG. 13, the protrusion 10″ includes bulges or pimples 10f that are arranged in a triangular manner, substantially at the center of the wall portion 9a″. It shall be noted that the protrusion 10″ with any of various other structures may also be implemented, for example, with structures similar to the protrusions shown in FIGS. 6 to 9.

FIG. 14 shows a first clinical application where the screw element 1 according to the first embodiment is screwed with the screwdriver 20, the engagement portion 30 of which engages the drive portion 6, into the pedicle of a vertebra 500. In FIG. 15, a polyaxial bone anchoring device is shown that may include the screw element 1 with the drive portion 6, 6′ according to the first embodiment. For fixing the rod 200, set screw 40 according to the second embodiment may be used. The polyaxial bone anchoring device may be a bottom-loading polyaxial bone anchoring device, i.e., where the head 4, 4′ of the screw element 1 can be inserted into the receiving part from the bottom of the receiving part 51. Thus, the screw element 1 can be inserted first into the bone as shown in FIG. 14, and the receiving part 51 can be mounted thereafter. The drive portion 6, 6′ according to the screw element 1 of the first embodiment is particularly useful for such in-situ placement of the screw element 1 into the vertebra prior to mounting the receiving part 51. However, the screw element 1 according to the first embodiment can also be preassembled with the receiving part prior to insertion into bone.

A further clinical application is shown in FIG. 16. FIG. 16 depicts a bone plate 60 that may be used with the screw element 1 of the first embodiment shown in FIGS. 1 to 9, for example, in orthopaedic and trauma surgery, to immobilize broken bones and/or bone parts. The spherical head 4, 4′ of the screw element 1 can assume, within a hole 61 of the bone plate 60, various angles. Alternatively, the head and/or hole 61 may have a shape that limits positioning of the screw element 1, for example, to a fixed angle with respect to the bone plate 60. When implanting the bone plate 60, the screwdriver 20 engages the drive portion 6, 6′ with the engagement portion 30 to fix the bone plate 60 with the screw element 1 to bone. In a still further modification, a locking element (not shown) in the form of a set screw may be provided in the holes of the bone plate 60, for example, to prevent pulling out of the screw elements 1. Such a locking element may have a drive portion 6″ similar to those shown in FIGS. 10 to 13. Such a locking element may have, at its lower side, a recess for receiving a portion of the head 4, 4′ of the screw element 1.

Further modifications of the screw element are also conceivable. Without restriction, the following are examples of such possible modifications. The second recess 11 can be omitted. The bottom of the first recess 7, 7″ can be open or closed. For example, if the screw element 1 is a cannulated bone anchor, the bottom of the first recess may be open. In the embodiments shown, an even number of drive grooves 8, 8″ are shown, and each drive groove 8, 8″ is positioned opposite to another drive groove in the drive portions 6, 6′, 6″. However, an odd number of drive grooves may also be contemplated. The design of the protrusion or protrusions may vary between the wall portions within one drive portion.

In addition, instead of the star-shape or torx-shape of the first recess, a first recess having a polygonal shape may also be contemplated. In such cases, the corners of the polygon may be considered the drive grooves.

The screw element according to the first embodiment may also be formed as a bone nail. The head may have other external shapes instead of spherical, for example, cylindrical or otherwise shaped. The bone anchoring device can also be, for example, a mono-planar bone anchoring device where the screw element can pivot only in a single plane, instead of a polyaxial device.

The screw element of the second embodiment may also implement the designs of the drive portions shown in FIGS. 6 to 9, and the screw element of the first element may also implement the designs of the drive portions shown in FIGS. 11 to 13, etc.

While the present invention has been described in connection with certain exemplary embodiments, it is to be understood that the invention is not limited to the disclosed embodiments, but is instead intended to cover various modifications and equivalent arrangements included within the spirit and scope of the appended claims, and equivalents thereof.

What is claimed is:

1. A monolithic screw element for use in spinal, orthopaedic, or trauma surgery, the screw element having a screw axis and comprising:
a screw thread; and
a drive portion comprising a wall that defines an axially extending recess for engaging a tool, the recess having a cross-sectional profile that extends from a first end to a second end in a direction of the screw axis, wherein the cross-sectional profile has a first portion positioned closest radially to the screw axis and a plurality of drive grooves that extend radially away from the first portion and that are spaced apart from each other in a circumferential direction around the screw axis;
wherein a portion of the wall comprises a protrusion that protrudes laterally away from the wall into the recess, and wherein the protrusion is spaced apart axially from the first and second ends of the recess.

2. The screw element of claim 1, wherein the protrusion comprises a geometrical shape.

3. The screw element of claim 1, wherein the protrusion is formed by roughening a surface of the wall.

4. The screw element of claim 3, wherein the roughening is structured or organized.

5. The screw element of claim 1, wherein the protrusion is formed to be locally distinct on the wall.

6. The screw element of claim 1, wherein the protrusion comprises one or more bulges, pimples, and/or ribs.

7. The screw element of claim 1, wherein the protrusion extends circumferentially around at least part of the recess.

8. The screw element of claim 1, wherein the cross-sectional profile forms a star-like shape or a polygonal shape.

9. The screw element of claim 1, wherein the recess is a first recess, wherein the screw element defines a second recess between the first recess and a free end of the screw element, and wherein the second recess comprises guide grooves that are wider than the drive grooves and that are located at circumferential positions corresponding to circumferential positions of the drive grooves.

10. The screw element of claim 1, wherein the screw element comprises a bone screw comprising a shank on which the screw thread is formed on at least a portion thereof, and a head having a free end opposite the shank, and wherein the drive portion is formed at the free end of the head.

11. The screw element of claim 1, wherein the screw element comprises a set screw configured to lock a polyaxial bone anchoring device or a bone plate.

12. The screw element of claim 1, wherein the protrusion is formed on a portion of the wall that defines the first portion of the cross-sectional profile.

13. The screw element of claim 1, wherein the protrusion protrudes radially inwardly from the wall towards the screw axis.

14. A system comprising:
a screw element for use in spinal, orthopaedic, or trauma surgery, the screw element having a screw axis and comprising a screw thread and a drive portion comprising a wall that defines an axially extending recess for engaging a tool, the recess having a cross-sectional profile that extends from a first end to a second end in a direction of the screw axis, wherein the cross-sectional profile has a first portion positioned closest radially to the screw axis and a plurality of drive grooves that extend radially away from the first portion and that are spaced apart from each other in a circumferential direction around the screw axis, wherein a portion of the wall comprises a protrusion that protrudes laterally away from the wall into the recess; and
a tool having a longitudinal axis and comprising an engagement portion for engaging the recess, wherein the engagement portion has a cross-sectional profile that corresponds substantially to the cross-sectional profile of the recess and that extends from a first end to a second end in a direction of a longitudinal axis;
wherein when the engagement portion of the tool is in the recess of the screw element with the region between the first and second ends of the engagement portion being positioned at a same axial height as a region between the first and second ends of the recess, the engagement portion is configured to engage the protrusion, while at least part of the wall of the screw element positioned axially between the protrusion and the first end of the recess and at least part of the wall of the screw element positioned axially between the protrusion and the second end of the recess are both configured to be spaced apart from the engagement portion in a lateral direction.

15. The system of claim 14, wherein the tool configured to engage the recess of the drive portion comprises a screwdriver.

16. The system of claim 14, wherein the protrusion is sized to reduce lateral movement of the engagement portion when the engagement portion is in the recess.

17. The system of claim 14, wherein the protrusion is configured to clamp the engagement portion by friction when the engagement portion is in the recess.

18. The system of claim 14, wherein the recess forms a spiral-like shape, with an inner portion of the wall that is closer to the screw axis and an outer portion of the wall that is farther away from the screw axis together defining the recess, and wherein the protrusion is formed on the inner portion of the wall.

19. The system of claim 14, wherein the protrusion is fixed relative to the wall.

20. A method of manufacturing a screw element for use in spinal orthopaedic, or trauma surgery, the screw element having a screw axis and comprising a screw thread and a drive portion comprising a wall that defines an axially extending recess for engaging a tool, the recess having a cross-sectional profile that extends from a first end to a second end in a direction of the screw axis, wherein the cross-sectional profile has a first portion positioned closest radially to the screw axis and a plurality of drive grooves that extend radially away from the first portion and that are spaced apart from each other in a circumferential direction around the screw axis, wherein a portion of the wall comprises a protrusion that protrudes laterally away from the wall into the recess, the method comprising:
providing computer data of a three-dimensional (3D) model of at least a first part of the screw element comprising the drive portion;
utilizing additive manufacturing to manufacture at least the first part of the screw element, such that at least the first part of the screw element is formed layer by layer from a building material according to a cross-section of the portion of the screw element corresponding to each respective layer.

21. The method of claim 20, wherein the additive manufacturing comprises at least one of laser sintering, laser melting, or electron beam melting.

22. The method of claim 20, wherein at least the drive portion of the screw element is sized and/or shaped to only be manufacturable by additive manufacturing.
